SEQUENCE LISTING (4 sequences)

SEQ ID NO: 1 - DNA (cDNA to mRNA), 2428 bp, Homo sapiens; double-stranded, linear
  CDS 82..2319: /product= “bile-salt-stimulated lipase”
  exon 985..1173
  exon 1174..1377
  exon 1378..1575
  exon 1576..2415
  mat_peptide 151..2316
  polyA_signal 2397..2402
  repeat_region 1756..2283
  5′UTR 1..81
  repeat_unit 1756..1788
  repeat_unit 1789..1821
  repeat_unit 1822..1854
  repeat_unit 1855..1887
  repeat_unit 1888..1920
  repeat_unit 1921..1953
  repeat_unit 1954..1986
  repeat_unit 1987..2019
  repeat_unit 2020..2052
  repeat_unit 2053..2085
  repeat_unit 2086..2118
  repeat_unit 2119..2151
  repeat_unit 2152..2184
  repeat_unit 2185..2217
  repeat_unit 2218..2250
  repeat_unit 2251..2283
  hypothetical: NO
  anti-sense: NO
  original source tissue type: mammary gland
  authors: Jeanette Blackberg, Lars Carlsson, Peter Enerback, Sven Hernell, Olle Bjursell, Gunnar Nilsson
  title: cDNA cloning of human-milk bile-salt-stimulated lipase and evidence for its identity to pancreatic carboxylic ester hydrolase
  journal: Eur. J. Biochem.
  volume: 192
  pages: 543-550
  date: Sept.-1990
accttctgtatcagttaagtgtcaagatggaaggaacagcagtctcaagataatgcaaagagtttattcatccagaggctgatgctcaccatggggcgcctgcaactggttgtgttgggcctcacctgctgctgggcagtggcgagtgccgcgaagctgggcgccgtgtacacagaaggtgggttcgtggaaggcgtcaataagaagctcggcctcctgggtgactctgtggacatcttcaagggcatccccttcgcagctcccaccaaggccctggaaaatcctcagccacatcctggctggcaagggaccctgaaggccaagaacttcaagaagagatgcctgcaggccaccatcacccaggacagcacctacggggatgaagactgcctgtacctcaacatttgggtgccccagggcaggaagcaagtctcccgggacctgcccgttatgatctggatctatggaggcgccttcctcatggggtccggccatggggccaacttcctcaacaactacctgtatgacggcgaggagatcgccacacgcggaaacgtcatcgtggtcaccttcaactaccgtgtcggcccccttgggttcctcagcactggggacgccaatctgccaggtaactatggccttcgggatcagcacatggccattgcttgggtgaagaggaatatcgcggccttcgggggggaccccaacaacatcacgctcttcggggagtctgctggaggtgccagcgtctctctgcagaccctctccccctacaacaagggcctcatccggcgagccatcagccagagcggcgtggccctgagtccctgggtcatccagaaaaacccactcttctgggccaaaaaggtggctgagaaggtgggttgccctgtgggtgatgccgccaggatggcccagtgtctgaaggttactgatccccgagccctgacgctggcctataaggtgccgctggcaggcctggagtaccccatgctgcactatgtgggcttcgtccctgtcattgatggagacttcatccccgctgacccgatcaacctgtacgccaacgccgccgacatcgactatatagcaggcaccaacaacatggacggccacatcttcgccagcatcgacatgcctgccatcaacaagggcaacaagaaagtcacggaggaggacttctacaagctggtcagtgagttcacaatcaccaaggggctcagaggcgccaagacgacctttgatgtctacaccgagtcctgggcccaggacccatcccaggagaataagaagaagactgtggtggactttgagaccgatgtcctcttcctggtgcccaccgagattgccctagcccagcacagagccaatgccaagagtgccaagacctacgcctacctgttttcccatccctctcggatgcccgtctaccccaaatgggtgggggccgaccatgcagatgacattcagtacgttttcgggaagcccttcgccacccccacgggctaccggccccaagacaggacagtctctaaggccatgatcgcctactggaccaactttgccaaaacaggggaccccaacatgggcgactcggctgtgcccacacactgggaaccctacactacggaaaacagcggctacctggagatcaccaagaagatgggcagcagctccatgaagcggagcctgagaaccaacttcctgcgctactggaccctcacctatctggcgctgcccacagtgaccgaccaggaggccacccctgtgccccccacaggggactccgaggccactcccgtgccccccacgggtgactccgagaccgcccccgtgccgcccacgggtgactccggggccccccccgtgccgcccacgggtgactccggggccccccccgtgccgcccacgggtgactccggggccccccccgtgccgcccacgggtgactccggggccccccccgtgccgcccacgggtgactccggggccccccccgtgccgcccacgggtgactccggggccccccccgtgccgcccacgggtgactccggcgccccccccgtgccgcccacgggtgacgccgggcccccccccgtgccgcccacgggtgactccggcgccccccccgtgccgcccacgggtgactccggggccccccccgtgacccccacgggtgactccgagaccgcccccgtgccgcccacgggtgactccggggccccccctgtgccccccacgggtgactctgaggctgcccctgtgccccccacagatgactccaaggaagctcagatgcctgcagtcattaggttttagcgtcccatgagccttggtatcaagaggccacaagagtgggaccccaggggctcccctcccatcttgagctcttcctgaataaagcctcatacccctaaaaaaaaaaaaa

SEQ ID NO: 2 - protein, 745 aa
MLTMGRLQLVVLGLTCCWAVASAAKLGAVYTEGGFVEGVNKKLGLLGDSVDIFKGIPFAAPTKALENPQPHPGWQGTLKAKNFKKRCLQATITQDSTYGDEDCLYLNIWVPQGRKQVSRDLPVMIWIYGGAFLMGSGHGANFLNNYLYDGEEIATRGNVIVVTFNYRVGPLGFLSTGDANLPGNYGLRDQHMAIAWVKRNIAAFGGDPNNITLFGESAGGASVSLQTLSPYNKGLIRRAISQSGVALSPWVIQKNPLFWAKKVAEKVGCPVGDAARMAQCLKVTDPRALTLAYKVPLAGLEYPMLHYVGFVPVIDGDFIPADPINLYANAADIDYIAGTNNMDGHIFASIDMPAINKGNKKVTEEDFYKLVSEFTITKGLRGAKTTFDVYTESWAQDPSQENKKKTVVDFETDVLFLVPTEIALAQHRANAKSAKTYAYLFSHPSRMPVYPKWVGADHADDIQYVFGKPFATPTGYRPQDRTVSKAMIAYWTNFAKTGDPNMGDSAVPTHWEPYTTENSGYLEITKKMGSSSMKRSLRTNFLRYWTLTYLALPTVTDQEATPVPPTGDSEATPVPPTGDSETAPVPPTGDSGAPPVPPTGDSGAPPVPPTGDSGAPPVPPTGDSGAPPVPPTGDSGAPPVPPTGDSGAPPVPPTGDSGAPPVPPTGDAGPPPVPPTGDSGAPPVPPTGDSGAPPVTPTGDSETAPVPPTGDSGAPPVPPTGDSEAAPVPPTDDSKEAQMPAVIRF

SEQ ID NO: 3 - protein, 722 aa, Homo sapiens
  hypothetical: NO
  original source tissue type: Mammary gland
AKLGAVYTEGGFVEGVNKKLGLLGDSVDIFKGIPFAAPTKALENPQPHPGWQGTLKAKNFKKRCLQATITQDSTYGDEDCLYLNIWVPQGRKQVSRDLPVMIWIYGGAFLMGSGHGANFLNNYLYDGEEIATRGNVIVVTFNYRVGPLGFLSTGDANLPGNYGLRDQHMAIAWVKRNIAAFGGDPNNITLFGESAGGASVSLQTLSPYNKGLIRRAISQSGVALSPWVIQKNPLFWAKKVAEKVGCPVGDAARMAQCLKVTDPRALTLAYKVPLAGLEYPMLHYVGFVPVIDGDFIPADPINLYANAADIDYIAGTNNMDGHIFASIDMPAINKGNKKVTEEDFYKLVSEFTITKGLRGAKTTFDVYTESWAQDPSQENKKKTVVDFETDVLFLVPTEIALAQHRANAKSAKTYAYLFSHPSRMPVYPKWVGADHADDIQYVFGKPFATPTGYRPQDRTVSKAMIAYWTNFAKTGDPNMGDSAVPTHWEPYTTENSGYLEITKKMGSSSMKRSLRTNFLRYWTLTYLALPTVTDQEATPVPPTGDSEATPVPPTGDSETAPVPPTGDSGAPPVPPTGDSGAPPVPPTGDSGAPPVPPTGDSGAPPVPPTGDSGAPPVPPTGDSGAPPVPPTGDSGAPPVPPTGDAGPPPVPPTGDSGAPPVPPTGDSGAPPVTPTGDSETAPVPPTGDSGAPPVPPTGDSEAAPVPPTDDSKEAQMPAVIRF

SEQ ID NO: 4 - protein, 568 aa, Homo sapiens
  Peptide 1..568: /label= Variant_C
  hypothetical: NO
  original source tissue type: Mammary gland
  authors: Lennart Blackberg, Lars Edlund, Michael Lundberg, Lennart Stromqvist, Mats Hernell, Olle Hansson
  title: Recombinant Human Milk Bile Salt-stimulated Lipase
  journal: J. Biol. Chem.
  volume: 268
  issue: 35
  pages: 26692-26698
  date: Dec. 15-1993
AKLGAVYTEGGFVEGVNKKLGLLGDSVDIFKGIPFAAPTKALENPQPHPGWQGTLKAKNFKKRCLQATITQDSTYGDEDCLYLNIWVPQGRKQVSRDLPVMIWIYGGAFLMGSGHGANFLNNYLYDGEEIATRGNVIVVTFNYRVGPLGFLSTGDANLPGNYGLRDQHMAIAWVKRNIAAFGGDPNNITLFGESAGGASVSLQTLSPYNKGLIRRAISQSGVALSPWVIQKNPLFWAKKVAEKVGCPVGDAARMAQCLKVTDPRALTLAYKVPLAGLEYPMLHYVGFVPVIDGDFIPADPINLYANAADIDYIAGTNNMDGHIFASIDMPAINKGNKKVTEEDFYKLVSEFTITKGLRGAKTTFDVYTESWAQDPSQENKKKTVVDFETDVLFLVPTEIALAQHRANAKSAKTYAYLFSHPSRMPVYPKWVGADHADDIQYVFGKPFATPTGYRPQDRTVSKAMIAYWTNFAKTGDPNMGDSAVPTHWEPYTTENSGYLEITKKMGSSSMKRSLRTNFLRYWTLTYLALPTVTDQGAPPVPPTGDSGAPPVPPTGDSKEAQMPAVIRF

(19) United States
(12) Patent Application Publication
(10) Pub. No.: US 2003/0040040 A1
(43) Pub. Date: Feb. 27, 2003

(54) DNA MOLECULES FOR EXPRESSION OF BILE SALT-STIMULATED LIPASE

(76) Inventor: GOUTAM DAS, BANGALORE (IN)

Correspondence Address: WHITE & CASE LLP, PATENT DEPARTMENT, 1155 AVENUE OF THE AMERICAS, NEW YORK, NY, 10036, US

(21) Appl. No.: 09/418,176

(22) Filed: Oct. 13, 1999

Related U.S. Application Data
(63) Continuation of application No. 08/624,398, filed on Apr. 4, 1996, now abandoned.
Application No. 08/624,398 is a 371 of international application No. PCT/SE96/00318, filed on Mar. 12, 1996.

(30) Foreign Application Priority Data
Mar. 23, 1995 (IN) 351/MAS/95
Jun. 24, 1995 (SE) 9501939-4

(51) Int. Cl. C07H 21/04; C12N 9/00; C12N 15/00; C12P 21/02; C12N 5/06; C12N 9/20

(52) U.S. Cl. 435/69.1; 435/320.1; 435/325; 435/183; 536/23.2

(57) ABSTRACT

The invention relates to DNA molecules, recombinant vectors and cell cultures for use in methods for expression of bile salt-stimulated lipase (BSSL) in the methylotrophic yeast Pichia pastoris.

### TECHNICAL FIELD

The invention relates to DNA molecules, recombinant vectors and cell cultures for use in methods for expression of bile salt-stimulated lipase (BSSL) in the methylotrophic yeast Pichia pastoris.

### BACKGROUND ART

Bile salt-stimulated lipase (BSSL; EC 3.1.1.1) (for a review see Wang & Hartsuck, 1993) accounts for the majority of the lipolytic activity of the human milk. A characteristic feature of this lipase is that it requires primary bile salts for activity against emulsified long chain triacylglycerols. BSSL has so far been found only in milk from man, gorilla, cat and dog (Hernell et al., 1989).

BSSL has been attributed a critical role for the digestion of milk lipids in the intestine of the breastfed infant (Fredrikzon et al., 1978). BSSL is synthesized in humans in the lactating mammary gland and secretes with milk (Bläckberg et al., 1987). It accounts for approximately 1% of the total milk protein (Bläckberg & Hernell, 1981).

It has been suggested that BSSL is the major rate limiting factor in fat absorption and subsequent growth by, in particular premature, infants who are deficient in their own production of BSSL, and that supplementation of formulas with the purified enzyme significantly improves digestion and growth of these infants (U.S. Pat. No. 4,944,944; Oklahoma Medical Research Foundation). This is clinically important in the preparation of infant formulas which contain relative high percentage of triglycerides and which are based on plant or non human milk protein sources, since infants fed with these formulas are unable to digest the fat in the absence of added BSSL.

The cDNA structures for both milk BSSL and pancreas carboxylic ester hydrolase (CEH) have been characterized (Baba et al., 1991; Hui and Kissel, 1991; Nilsson et al., 1991; Reue et al., 1991) and the conclusion has been drawn that the milk enzyme and the pancreas enzyme are products of the same gene, the CEL gene. The cDNA sequence (SEQ ID NO: 1) of the CEL gene is disclosed in U.S. Pat. No. 5,200,183 (Oklahoma Medical Research Foundation); WO 91/18293 (Aktiebolaget Astra); Nilsson et al., (1990); and Baba et al., (1991). The deduced amino acid sequence of the BSSL protein, including a signal sequence of 23 amino acids, is shown as SEQ ID NO: 2 in the Sequence Listing, while the sequence of the native protein of 722 amino acids is shown as SEQ ID NO: 3.

The C-terminal region of the protein contains 16 repeats of 11 amino acid residues each, followed by an 11 amino acid conserved stretch. The native protein is highly glycosylated and a large range of observed molecular weights have been reported. This can probably be explained by varying extent of glycosylation (Abouakil et al., 1988). The N-tenninal half of the protein is homologous to acetyl choline esterase and some other esterases (Nilsson et al., 1990).

Recombinant BSSL can be produced by expression in a suitable host such as E. coli, Saccharomyces cerevisiae, or mammalian cell lines. For the scaling-up of a BSSL expression system to make the production cost commercially viable, utilization of heterologous expression systems could be envisaged. As mentioned above, human BSSL has 16 repeats of 11 amino acids at the C-terminal end. To determine the biological significance of this repeat region, various mutants of human BSSL have been constructed which lack part or whole of the repeat regions (Hansson et al., 1993). The variant BSSL-C (SEQ ID NO: 4), for example, has deletions from amino acid residues 536 to 568 and from amino acid residues 591 to 711. Expression studies, using mammalian cell line C127 host and bovine papilloma virus expression vector, showed that the various variants can be expressed in active forms (Hansson et al., 1993). From the expression studies it was also conduded that the proline rich repeats in human BSSL are not essential for catalytic activity or bile salt activation of BSSL. However, production of BSSL or its mutants in a mammalian expression system could be too expensive for routine therapeutic use.

A eukaryotic system such as yeast may provide significant advantages, compared to the use of prokaryotic systems, for the production of certain polypeptides encoded by recombinant DNA. For example, yeast can generally be grown to higher cell densities than bacteria and may prove capable of glycosylating expressed polypeptides, where such glycosylation is important for the biological activity. However, use of the yeast Saccharomyces cerevisiae as a host organism often leads to poor expression levels and poor secretion of the recombinant protein (Cregg et al., 1987). The maximum levels of heterologous proteins in S. cerevisae are in the region of 5% of total cell protein (Kingsman et al., 1985). A further drawback of using Sacharomyces cerevisiae as a host is that the recombinant proteins tend to be overglycosylated which could affect activity of glycosylated mammalian proteins.

Pichia pastoris is a methylotrophic yeast which can grow on methanol as a sole carbon and energy source as it contains a highly regulated methanol utilization pathway (Ellis et al., 1985). P. pastoris is also amenable to efficient high cell density fermentation technology. Therefore recombinant DNA technology and efficient methods of yeast transformation have made it possible to develop P. pastoris as a host for expression of heterologous protein in large quantity, with a methanol oxidase promoter based expression system (Cregg et al., 1987).

Use of Pichia pastoris is known in the art as a host for the expression of e.g. the following heterologous proteins: human tumor necrosis factor (EP-A-0263311); Bordetella pertactin antigens (WO 91/15571); hepatitis B surface antigen (Cregg et al., 1987); human lysozyme protein (WO 92/04441); aprotinin (WO 92/01048). However, successful expression of a heterologous protein in active, soluble and secreted form depends on a variety of factors, e.g. correct choice of signal peptide, proper construction of the fusion junction between the signal peptide and the mature protein, growth conditions, etc.

### PURPOSE OF THE INVENTION

The purpose of the invention is to overcome the above mentioned drawbacks with the previous systems and to provide a method for the production of human BSSL with is cost-effective and has a yield comparable with, or superior to, production in other organisms. This purpose has been achieved by providing methods for expression of BSSL in Pichia pastoris cells.

By the invention it has thus been shown that human BSSL and the variant BSSL can be expressed in active form secreted from P. pastoris. The native signal peptide, as well as the heterologous signal peptide derived from S. cerevisiae invertase protein, have been used to transiocate the mature protein into the culture medium as an active, properly processed form.

### DESCRIPTION OF THE INVENTION

In a first aspect, the invention provides a DNA molecule comprising:

(a) a region coding for a polypeptide which is human BSSL or a biologically active variant thereof;

(b) joined to the 5′-end of said polypeptide coding region, a region coding for a signal peptide capable of directing secretion of said polypeptide from Pichia pastoris cells transformed with said DNA molecule; and

(c) operably-linked to said coding regions defined in (a) and (b), the methanol oxidase promoter of Pichia pastoris or a functionally equivalent promoter.

The term “biologically active variant” of BSSL is to be understood as a polypeptide having BSSL activity and comprising part of the amino acid sequence shown as SEQ ID NO: 3 in the Sequence Listing. The term “polypeptide having BSSL activity” is in this context to be understood as a polypeptide comprising the following properties: (a) being suitable for oral administration; (b) being activated by specific bile-salts; and (c) acting as a non-specific lipase in the contents of the small intestines, i.e. being able to hydrolyze lipids relatively independent of their chemical structure and physical state (emulsified, micellar, soluble).

The said BSSL variant can e.g. be a variant which comprises less than 16 repeat units, whereby a “repeat unit” will be understood as a repeated unit of 11 amino acids, encoded by a nudeotide sequence indicated as a “repeat unit” under the heading “(ix) FEATURE” in “INFORMATION FOR SEQ ID NO: 1” in the Sequence Listing. In particular, the BSSL variant can be the variant BSSL-C, wherein amino acids 536 to 568 and 591 to 711 have been deleted (SEQ ID NO: 4 in the Sequence Listing). Consequently, the DNA molecule according to the invention is preferably a DNA molecule which encodes BSSL (SEQ ID NO: 3) or BSSL-C (SEQ ID NO: 4).

However, the DNA molecules according to the invention are not to be limited strictly to DNA molecules which encode polypeptides with amino acid sequences identical to SEQ ID NO: 3 or 4 in the Sequence Listing. Rather the invention encompasses DNA molecules which code for polypeptides carrying modifications like substitutions, small deletions, insertions or inversions, which polypeptides nevertheless have substantially the biological activities of BSSL. Included in the invention are consequently DNA molecules coding for BSSL variants as stated above and also DNA molecules coding for polypeptides, the amino acid sequence of which is at least 90% homologous, preferably at least 95% homologous, with the amino acid sequence shown as SEQ ID NO: 3 or 4 in the Sequence Listing.

The signal peptide referred to above can be a peptide which is identical to, or substantially similar to, the peptide with the amino acid sequence shown as amino acids −20 to −1 of SEQ ID NO: 2 in the Sequence Listing. Alternatively, it can be a peptide which comprises a Saccharomyces cerevisiae invertase signal peptide.

In a further aspect, the invention provides a vector comprising a DNA molecule as defined above. Preferably, such a vector is a replicable expression vector which carries and is capable of mediating expression, in a cell of the genus Pichia, of a DNA sequence coding for human BSSL or a biologically active variant thereof. Such a vector can e.g. be the plasmid vector pARC 5771 (NCIMB 40721), pARC 5799 (NCIMB 40723) or pARC 5797 (NCIMB 40722).

In another aspect, the invention provides a host cell culture comprising cells of the genus Pichia transformed with a DNA molecule or a vector as defined above. Preferably, the host cells are Pichia pastoris cells of a strain such as PPF-1 or GS115. The said cell culture can e.g. be the culture PPF-1[pARC 5771] (NCIMB 40721), GS115[pARC 5799] (NCIMB 40723) or GS115[pARC 5797] (NCIMB 40722).

In yet another aspect, the invention provides a process the production of a polypeptide which is human BSSL, or a biologically active variant thereof, which comprises culturing host cells according to the invention under conditions whereby said polypeptide is secreted into the culture medium, and recovering said polypeptide from the culture medium.

### EXAMPLES OF THE INVENTION

### Example 1

### Expression of BSSL in Pichia pastoris PPF-1

1.1. Construction of pARC 0770

The cDNA sequence (SEQ ID NO: 1) coding for the BSSL protein, including the native signal peptide (below referred to as NSP) was cloned in pTZ19R (Pharmacia) as an EcoRI-SacI fragment. The cloning of NSP-BSSL cDNA into S. cerevisiae expression vector pSCW 231 (obtained from professor L. Prakash, University of Rochester, N.Y., USA), which is a low copy number yeast expression vector wherein expression is under control of the constitutive ADH1 promoter, was achieved in two steps. Initially the NSP-BSSL cDNA was cloned into pYES 2.0 (Invitrogen, USA) as an EcoRI-SphI fragment from pTZ19R-SP-BSSL. The excess 89 base pairs between the EcoRI and NcoI at the beginning of the signal peptide coding sequence were removed by creating an EcoRI/NcoI (89) fusion and regenerating an EcoRI site. The resulting clone pARC 0770 contained an ATG codon, originally encoded within the NcoI site which was immediately followed by the regenerated EcoRI site in frame with the remaining NSP-BSSL sequence.

1.2. Construction of pARC 5771 Plasmid

To construct a suitable expression vector for the expression of BSSL, the cDNA fragment encoding the BSSL protein along with its native signal peptide was cloned with P. pastoris expression vector pDM 148. The vector pDM 148 (received from Dr. S. Subramani, UCSD) was constructed as follows: the upstream untranslated region (5′-UTR) and the down stream untranslated region (3′-UTR) of methanol oxidase (MOX1) gene were isolated by PCR and placed in tandem in the multiple cloning sequence (MCS) of E. coli vector pSK⁺ (available from Stratagene, USA).

For proper selection of the putative P. pastoris transformants, a DNA sequence coding for S. cerevisiae ARG4 gene along with its own promoter sequence was inserted between the 5′- and the 3′-UTR in pSK−. The resulting construct pDM148 has following features: in the MCS region of pSK− the 5′-UTR of MOX, S. cerevisiae ARG4 genomic sequence and the 3′-UTR of MOX were cloned. Between the 5′-UTR of MOX and the ARG4 genomic sequence a series of unique restriction sites (SalI, ClaI, EcoRI, PstI, SmaI and BamHI) were situated where any heterologous protein coding sequence can be cloned for expression under the control of the MOX promoter in P. pastoris. To facilitate integration of this expression cassette into the MOX1 locus in P. pastoris chromosome, the expression cassette can be cleaved from the rest of the pSK⁻ vector by digestion with NotI restriction enzyme.

The 5′-UTR of MOX1 of P. pastoris cloned in pDM 148 was about 500 bp in length while the 3′-UTR of MOX1 from P. pastoris cloned into pDM 148 was about 1000 bp long. To insert the NSP-BSSL cDNA sequence, between the 5′-UTR of MOX1 and the S. cerevisiae ARG4 coding sequence in pDM 148, the cDNA insert (SP-BSSL) was isolated from pARC 0770 by digestion with EcoRI and BamHI (approximately 2.2 kb DNA fragment) and cloned between the EcoRI and BamHI sites in pDM 148.

The resulting construct pARC 5771 (NCIMB 40721) contained the P. pastoris MOX1 5′-UTR followed by the NSP-BSSL coding sequence followed by S. cerevisiae ARG4 gene sequence and 3′-UTR of MOX1 gene of P. pastoris while the entire DNA segment from 5′-UTR of MOX1 to the 3′-UTR of MOX1 was cloned at the MCS of pSK−.

1.3. Transformation of BSSL in P. pastoris Host PPF-1

For expression of BSSL in P. pastoris host PPF-1 (his4, arg4; received from Phillips Petroleum Co.), the plasmid pARC 5771 was digested with NotI and the entire digested mix (10 μg of total DNA) was used to transform PPF-1. The transformation protocol followed was essentially the yeast spheroplast method described by Cregg et al. (1987). Transformants were regenerated on minimal medium lacking arginine so that Arg+ colonies could be selected. The regeneration top agar containing the transformants was lifted and homogenized in water and yeast cells plated to about 250 colonies per plate on minimal glucose plates lacking arginine. Mutant colonies are then identified by replica plating onto minimal methanol plates. Approximately 15% of all transformants turned out to be Mut^{s} (methanol slow growing) phenotype.

1.4. Screening for Transformants Expressing BSSL

In order to screen large number of transformants rapidly for the expression of lipase a lipase plate assay method was developed. The procedure for preparing these plates was as follows: to a solution of 2% agarose (final), 10×Na-cholate solution in water was added to a final concentration of 1%. The lipid substrate trybutine was added in the mixture to a final concentration of 1% (v/v). To support growth of the transformants the mixture was further supplemented with 0.25% yeast nitrogen base (final) and 0.5% methanol (final). The ingredients were mixed properly and poured into plates up to 3-5 mm thickness. Once the mixture became solid, the transformants were streaked onto the plates and the plates were further incubated at +37° C. for 12 h. The lipase producing clones showed a clear halo around the clone. In a typical experiment 7 out of a total of 93 transformants were identified as BSSL producing transformants. Two clones (Nos. 39 and 86) producing the largest halos around the streaked colony were picked out for further characterization.

1.5. Expression of BSSL from PPF-1[pARC 5771]

The two transformants Nos. 39 and 86 described in Section 1.4 were picked out and grown in BMGY liquid media (1% yeast extract, 2% bactopeptone, 1.34% yeast nitrogen base without amino acid, 100 mM KPO₄ buffer, pH 6.0, 400 μg/l biotin, and 2% glycerol) for 24 h at 30° C. until the cultures reached A₆₀₀ close to 40. The cultures were pelleted down and resuspended in BMMY (2% glycerol replaced by 0.5% methanol in BMGY) media at A₆₀₀=300. The induced cultures were incubated at 30° C. with shaking for 120 h. The culture supernatants were withdrawn at different time points for the analysis of the expression of BSSL by enzyme activity assay, SDS-PAGE analysis and western blotting.

1.6. Detection of BSSL Enzyme Activity in the Culture Supernatants of Clone Nos. 39 and 86

To determine the enzyme activity in the cell free culture supernatant of the induced cultures Nos. 39 and 86 as described in Section 1.5, the cultures were spun down and 2 μl of the cell free supernatant was assayed for BSSL enzyme activity according to the method described by Hernell and Olivecrona (1974). As shown in Table 1, both the cultures were found to contain BSSL enzyme activity with the maximum activity at 96 h following induction.

1.7. Western Blot Analysis of Culture Supernatants of PPF-1:pARC 5771 Transformants (Nos. 39 and 86)

To determine the presence of recombinant BSSL in the culture supernatants Nos. 39 and 86 of PPF-1[pARC 5771] transformants, the cultures were grown and induced as described in Section 1.5. The cultures were withdrawn at different time points following induction and subjected to Western blot analysis using anti BSSL polyclonal antibody. The results indicated the presence of BSSL in the culture supernatant as a 116 kDa band.

### Example 2

### Expression of BSSL in Pichia pastoris GS115

2.1. Construction of pARC 5799

Since the 5′-MOX UTR and 3′-MOX UTR were not properly defined and since the pDM 148 vector lacks any other suitable marker (e.g. a G418 resistance gene) to monitor the number of copies of the BSSL integrated in the Pichia chromosome, the cDNA insert of native BSSL along with its signal peptide was cloned into another P. pastoris expression vector, pHIL D4. The integrative plasmid pHIL D4 was obtained from Phillips Petroleum Company. The plasmid contained 5′-MOX1, approximately 1000 bp segment of the alcohol oxidase promoter and a unique EcoRI doning site. It also contained approximately 250 bp of 3′-MOX1 region containing alcohol oxidase terminating sequence, following the EcoRI site. The “termination” region was followed by P. pastoris histidinol dehydrogenase gene HIS4 contained on a 2.8 kb fragment to complement the defective HIS4 gene in the host GS115 (see below). A 650 bp region containing 3′-MOX1 DNA was fused at the 3′-end of HIS4 gene, which together with the 5′-MOX1 region was necessary for site-directed integration. A bacterial kanamycin resistance gene from pUC4K (PL-Biochemicals) was inserted at the unique NaeI site between HIS4 and 3′-MOX1 region at 3′ of the HIS4 gene.

To clone the NSP-BSSL coding cDNA fragment at the unique EcoRI site of pHIL D4, a double stranded oligo linker having a BamHI-EcoRI cleaved position was ligated to the BamHI digested plasmid pARC 5771 and the entire NSP-BSSL coding sequence was pulled out as a 2.2 kb EcoRI fragment. This fragment was cloned at the EcoRI site of pHIL D-4 and the correctly oriented plasmid was designated as pARC 5799 (NCIMB 40723).

2.2. Transformation of pARC 5799

To facilitate integration of the NSP-BSSL coding sequence at the genomic locus of MOX1 in P. pastoris the plasmid pARC 5799 was digested with BglII and used for transformation of P. pastoris strain GS115(his4) (Phillips Petroleum Company) according to a protocol described in Section 1.5. In this case, however, the selection was for His prototrophy. The transformants were picked up following serial dilution plating of the regenerated top agar and tested directly for lipase plate assay as described in Section 1.4. Two transformant clones (Nos. 9 and 21) were picked up on the basis of the halo size on the lipase assay plate and checked further for the expression of BSSL. The clones were found to be Mut⁺.

2.3. Determination of BSSL Enzyme Activity in the Culture Supernatants of GS115[pARC 5799] Transformants Nos. 9 and 21.

The two transformed clones Nos. 9 and 21 of GS115[pARC 5799] were grown essentially following the protocol described in Section 1.5. The culture supernatants at different time points following induction were assayed for BSSL enzyme activity as described in Section 1.6. As shown in Table 1, both the culture supernatants were found to contain BSSL enzyme activity and the enzyme activity was highest after 72 h of induction. Both clones showed a superior expression of BSSL compared to the clones of PPF-1[pARC 5771].

2.4. SDS-PAGE and Western Blot Analysis of Culture Supernatants of GS115[pARC 5799] Transformants Nos. 9 and 21

The culture supernatants collected at different time points, as described in Section 2.3 were subjected to SDS-PAGE and western blot analysis. From the SDS-PAGE profile it was estimated that about 60-75% of the total protein present in the culture supernatants of the induced cultures was BSSL. The molecular weight of the protein was about 116 kDa. The western blot data also confirmed that the major protein present in the culture supernatant was BSSL. The protein apparently had the same molecular weight as the native BSSL.

### Example 3

### Scaling-Up of BSSL Expression

3.1. Scaling-up of Expression of BSSL from the Transformed Clone GS115[pARC 5799] (No. 21)

A 23 l capacity B. Braun fermenter was used. Five liters of medium containing, 1% YE, 2% Peptone, 1.34 YNB and 4% w/v glycerol was autoclaved at 121° C. for 30 mm and biotin (400 μg/L final concentration) was added during inoculation after filter sterilization. For inoculum, glycerol stock of GS115[pARC 5799] (No. 21) inoculated into a synthetic medium containing YNB (67%) plus 2% glycerol (150 ml) and grown at +30° C. for 36 h was used. Fermentation conditions were as follows: the temperature was +30° C.; pH 5.0 was maintained using 3.5 N NH₄OH and 2 N HCl; dissolved oxygen from 20 to 40% of air saturation; polypropylene glycol 2000 was used as antifoam.

Growth was monitored at regular intervals by taking OD at 600 nm. A₆₀₀ reached a maximum of 50-60 in 24 h. At this point, the batch growth phase was over as indicated by the increased dissolved oxygen levels.

Growth phase was immediately followed by the induction phase. During this phase, methanol containing 12 ml/L PTM1 salts was fed. Methanol feed rate was 6 μl/h during first 10-12 h after which it was increased gradually in 6 ml/h increments every 7-8 h to a maximum of 36 ml/h. Ammonia used for pH control acted as a nitrogen source. Methanol accumulation was checked every 6-8 h by using dissolved oxygen spiking and it was found to be limiting during the entire phase of induction. OD at 600 nm increased from 50-60 to 150-170 during 86 h of methanol feed. Yeast extract and peptone were added every 24 h to make final conc. of 0.25% and 0.5% respectively.

Samples were withdrawn at 24 h interval and checked for BSSL enzyme activity in the cell free broth. The broth was also subjected to SDS-PAGE and western blotting analysis.

3.2. Protein Analysis of the Secreted BSSL from the Fermenter Grown Culture GS115[pARC 5799] (No. 21)

BSSL enzyme activity in cell free broth increased from 40-70 mg/l (equivalent of native protein) in 24 h to a maximum 200-227.0 mg/l (equivalent of native protein) at the end of 86-90 h. SDS-PAGE analysis of the cell free broth shows a prominent coomassie blue stained band of mol.wt. of 116 kDa. The identity of the band was confirmed by Western blot performed as described in Section 1.7 for native BSSL.

3.3. Purification of Recombinant BSSL Secreted into the Culture Supernatant of GS115[pARC 5799] (No. 21) Clones

The P. pastoris clone GS115[pARC 5799] was grown and induced in the fermenter as described in Section 3.1. For purification of recombinant BSSL, 250 ml of culture medium (induced for 90 h) was spun at 12,000× g for 30 minutes to remove all particulate matter. The cell free culture supernatant was ultra filtered in an Amicon set up using a 10 kDa cut off membrane. Salts and low molecular weight proteins and peptides of the culture supernatant were removed by repeated dilution during filtration. The buffer used for such dilution was 5 mM Barbitol pH 7.4. Following concentration of the culture supernatant, the retentate was reconstituted to 250 ml using 5 mM Barbitol, pH 7.4 and 50 mM NaCl and loaded onto a Heparin-Sepharose column (15 ml bed volume) which was pre-equilibrated with the same buffer. The sample loading was done at a flow rate of 10 ml/hr. Following loading the column was washed with 5 mM Barbitol, pH 7.4 and 0.1 M NaCl (200 μl washing buffer) till the absorbance at 250 nm reached below detection level. The BSSL was eluted with 200 ml of Barbitol buffer (5 mM, pH 7.4) and a linear gradient of NaCl ranging from 0.1 M to 0.7 M. Fractions (2.5 ml) were collected and checked for the eluted protein by monitoring the absorbance at 260 nm. Fractions containing protein were assayed for BSSL enzyme activity. Appropriate fractions were analyzed on 8.0% SDS-PAGE to check thee purification profile.

3.4. Characterization of Purified Recombinant BSSL Secreted in the Culture Supernatant of GS115[pARC 5799]

SDS-PAGE and Western blot analysis of the fractions (described in Section 3.3) showing maximal BSSL enzyme activity demonstrated that the recombinant protein was approximately 90% pure. The molecular weight of the purified protein was about 116 kDa as determined by SDS-PAGE and western blot analysis. When the samples were overloaded for SDS-PAGE analysis a low molecular weight protein band could be detected by Coomassie Brilliant Blue staining which was not picked up on Western blot. The purified protein was subjected to N-terminal analysis in an automated protein sequencer. The results showed that the protein was properly processed from the native signal peptide and the recombinant protein has the N-terminal sequence A K L G A V Y. The specific activity of the purified recombinant protein was found to be similar to that of the native protein.

### Example 4

### Expression of BSSL-C in Pichia pastoris GS115

4.1. Construction of pARC 5797

The cDNA coding sequence for the BSSL variant BSSL-C was fused at its 5′-end with the signal peptide coding sequence of S. cerevisiae SUC2 gene product (invertase), maintaining the integrity of the open reading frame initiated at the first ATG codon of invertase signal peptide. This fusion gene construct was initially cloned into the S. cerevisiae expression vector pSCW 231 (pSCW 231 is a low copy number yeast expression vector and the expression is under the control of the constitutive ADH1 promoter) between EcoRI and BamHI site to generate the expression vector pARC 0788.

The cDNA of the fusion gene was further subdoned into P. pastoris expression vector pDM 148 (described in Section 1.2) by releasing the appropriate 1.8 kb fragment by EcoRI and BamHI digestion of pARC 0788 and subcloning the fragment into pDM 148 digested with EcoRI and BamHI. The resulting construct pARC 5790 was digested with BamHI. and a double stranded oligonudeotide linker of the physical structure BamHI-EcoRI-BamHI was ligated to generate the construct pARC 5796 essentially to isolate the cDNA fragment of the fusion gene, following the strategy as described in Section 2.1.

Finally the 1.8 kb fragment containing the invertase signal peptide/BSSL-C fusion gene was released from pARC 5796 by EcoRI digestion and cloned into pHIL D4 at the EcoRI site. By appropriate restriction analysis of the expression vector containing the insert in the proper orientation was identified and was designated as pARC 5797 (NCIMB 40722).

4.2. Expression of Recombinant BSSL-C from P. pastoris

To express recombinant BSSL-C from P. pastoris, the P. pastoris host GS115 was transformed with pARC 5797 by the method as described in Sections 1.3 and 2.2. Transformants were checked for lipase production by the method described in Sections 1.4 and 2.2. A single transformant (No. 3) was picked on the basis of high lipase producing ability by the lipase plate assay detection method and was further analyzed for production of BSSL enzyme activity in the culture supernatant by essentially following the method as described in Sections 1.6 and 2.3. As shown in Table 1, the culture supernatant of GS115[pARC 5797] (No. 3) contained BSSL enzyme activity and the amount increased progressively till 72 h following induction.

4.3. SDS-PAGE and Western Blot Analysis of Culture Supernatant of GS115[pARC 5797] Transformant (No. 3)

The culture supernatant collected at various time points as described in Section 4.2 were subjected to SDS-PAGE and western blot analysis as described in Sections 1.7 and 2.4. From the SDS-PAGE profile it was estimated that about 75-80% of the total extracellular protein was BSSLC. The molecular weight of the protein as estimated from SDS-PAGE analysis was approximately 66 kDa. On western blot analysis only two bands (doublet) around 66 kDa were found to be immunoreactive and thus confirming the expression of recombinant BSSL-C.

### Example for Comparison

### Expression of BSSL in S. cerevisiae

Attempts to express BSSL in Saccharomyces cerevisiae were made. BSSL was poorly secreted in S. cerevisiae and the native signal peptide did not work efficiently. In addition, the native signal peptide did not get cleaved from the mature protein in S. cerevisiae.

### REFERENCES

Abouakil, N., Rogalska, E., Bonicel, J. and Lombardo, D. (1988) Biochim. Biophys. Acta. 961, 299-308.

Baba, T., Downs, D., Jackson, K. W., Tang, J. and Wang, C -S (1991) Biochemistry 30, 500-510.

Bläckberg, L. and Hernell, O. (1981) Eur. J. Biochem. 116, 221-225.

Bläckberg, L., Ängquist, K. A. and Hernell, O. (1987) FEBS Lett. 217, 37-41.

Cregg, J. M. et al. (1987) Bio/Technology 5, 479-485.

Ellis, S. B. et al. (1985) Mol. Cell. Biol. 5, 1111-1121.

Fredrikzon, B., Hernell, O., Bläckberg, L. and Olivecrona, T. (1978) Pediatric Res. 12, 1048-1052.

Hansson, L., Bläckberg, L., Edlund, M., Lundberg, L., Strömqvist, M. and Hernell, O. (1993) J. Biol. Chem. 268, 26692-26698.

Hernell, O . and Olivecrona, T. (1974) Biochim. Biophys. Acta 369, 234-244.

Hernell, O., Bläckberg, L and Olivecrona, T. (1989) in: Textbook of gastroenterology and nutrition in infancy (Lebenthal, E., ed.) 347-354, Raven Press, NY.

Hernell, O. and Bläckberg, L. (1982) Pediatric Res. 16, 882-885.

Hui, D. Y. and Kissel, J. A. (1990) FEBS Letters 276, 131-134.

Kingsman, et. al. (1985) Biotechnology and Genetic Engineering Reviews 3, 377-416.

Nilsson, J., Bläckberg, L., Carlsson, P., Enerbäck, S., Hernell, O. and Bjursell, G. (1990) Eur. J. Biochem. 192, 543-550.

Reue, K., Zambaux, J., Wong, H., Lee, G., Leete, T. H., Ronk, M., Shively, J. E., Sternby, B., Borgström, B., Ameis, D. and Scholtz, M. C. (1991) J. Lipid. Res. 32, 267-276.

Wang, C-S, and Hartsuck, J. A. (1993) Biochim. Biphys Acta 1166, 1-19.

Deposit Of Microorganisms

The following plasmids, transformed into Pichia pastoris cultures, have been deposited under the Budapest Treaty at the National Collections of Industrial and Marine Bacteria (NCIMB), Aberdeen, Scotland, UK. The date of deposit is May 2, 1995.

| Strain[plasmid] | NCIMB No. |
|---|---|
| PPF-1[pARC 5771] | 40721 |
| GS115[pARC 5799] | 40723 |
| GS115[pARC 5797] | 40722 |

TABLE 1
Enzyme activity in the culture supernatants of Pichia pastoris
transformants.

| | Enzyme activity in mg/L equivalent of native BSSL | | | | |
|---|---|---|---|---|---|
| | PPF- | | GS115 | | |
| Hours after | 1[pARC 5771] | | [pARC 5799] | | GS115[pARC 5797] |
| induction | No.39 | No.86 | No.9 | No.21 | No.3 |
| 24 | 0.254 | 0.135 | 1.53 | 1.72 | 0.37 |
| 48 | 2.69 | 3.12 | 17.28 | 34.70 | 40.9 |
| 72 | 3.96 | 8.25 | 37.37 | 50.60 | 44.9 |
| 96 | 11.26 | 13.60 | 26.34 | 50.60 | 35.6 |
| 120 | 8.42 | 13.13 | 13.60 | 22.30 | 17.8 |

1. A DNA molecule comprising:
(a) a region coding for a polypeptide which is human BSSL or a biologically active variant thereof;
(b) joined to the 5′-end of said polypeptide coding region, a region coding for a signal peptide capable of directing secretion of said polypeptide from Pichia pastoris cells transformed with said DNA molecule; and
(c) operably-linked to said coding regions defined in (a) and (b), the methanol oxidase promoter of Pichia pastoris or a functionally equivalent promoter.

2. A DNA molecule according to claim 1 wherein the said signal peptide is identical to, or substantially similar to, the peptide with the amino acid sequence shown as amino acids −20 to −1 of SEQ ID NO: 2 in the Sequence Listing.

3. A DNA molecule according to claim 1 wherein the said signal peptide comp rises a Saccharomyces cerevisiae invertase signal peptide.

4. A DNA molecule according to any one of claims 1 to 3 encoding a biologically active variant of human BSSL in which at least one of the repeat units of 11 amino acids, said repeated units being indicated in SEQ ID NO: 1, is deleted.

5. A DNA molecule according to any one of claims 1 to 4 coding for a polypeptide which has BSSL activity and an amino acid sequence which is at least 95% homologous with the sequence according to SEQ ID NO: 3 or SEQ ID NO: 4.

6. A DNA molecule according to any one of claims 1 to 5 coding for a polypeptide which has the amino acid sequence according to SEQ ID NO: 3 or SEQ ID NO: 4.

7. A vector comprising a DNA molecule according to any one of claims 1 to 6.

8. A replicable expression vector according to claim 7 which is capable of mediating expression of human BSSL, or a biologically active variant thereof, in Pichia pastoris cells.

9. A vector according to claim 8 which is the plasmid vector pARC 5771 (NCIMB 40721), pARC 5799 (NCIMB 40723) or pARC 5797 (NCIMB 40722).

10. Host cells of the genus Pichia transformed with a vector according to any one of claims 7 to 9.

11. Host cells according to claim 10 which are Pichia pastoris cells.

12. Host cells according to claim 11 which are Pichia pastoris cells of the strain GS115.

13. Host cells according to claim 12 which are PPF-1[pARC 5771] (NCIMB 40721), GS115[pARC 5799] (NCIMB 40723) or GS115[pARC 5797] (NCIMB 40722).

14. A process for the production of a polypeptide which is human BSSL, or a biologically active variant thereof, which comprises culturing host cells according to any one of claims 10 to 13 under conditions whereby said polypeptide is secreted into the culture medium, and recovering said polypeptide from the culture medium.
